(19) United States
(12) Patent Application Publication
(10) Pub. No.: US 2008/0243082 A1
(43) Pub. Date: Oct. 2, 2008

(54) System for surgical drain fixation

(75) Inventor: Jack Goodman, Ann Arbor, MI (US)

Correspondence Address: BRINKS, HOFER, GILSON & LIONE, 2801 SLATER ROAD, SUITE 120, MORRISVILLE, NC, 27560, US

(73) Assignee: Closure Medical Corporation, Raleigh, NC (US)

(21) Appl. No.: 11/731,855

(22) Filed: Mar. 30, 2007

(51) Int. Cl. A61M 27/00

(52) U.S. Cl. 604/180

(57) ABSTRACT

Devices, systems and methods for fixation of medical articles which remain in a patient's body for a period of time are provided. The articles may be secured to the patient's skin by use of a device having an arcuate channel for holding the medical article. In one device, a base member including an arcuate channel receives the medical article, which base member has a plurality of protrusions which rest against the skin providing a gap for application of a polymerizable adhesive composition which polymerizes, forming a seal around the medical article and securing the medical article to the body. Alternatively, a device including a base member having an arcuate channel is provided adhered to a flexible member. The device receives the medical article and is positioned proximate to the patient's skin. Polymerizable adhesive composition is applied to the flexible member, securing the medical article to the patient's body upon polymerization.

### FIELD

The invention relates to systems, devices and methods for the securement or fixation of various medical articles during use.

### BACKGROUND

Presently, medical articles which extend into the body of the patient and exit the patient's body at some selected location on the body may be secured by passing sutures immediately adjacent to the point of exit of the medical article. Such medical articles secured by this method include surgical drains, for example. The ends of the suture are then tied around the shaft of the drain, locking it in place. Such means for securing surgical drains or other medical devices are known to cause discomfort related to the surgical drains which is a major source of patient complaint after a surgical procedure. The discomfort may be because if the drain is tugged in any fashion, the entire load is being borne by point sources on the skin (the points where the retaining sutures pass through).

Some designs for securing a medical article or device to or near a patient utilize pressure sensitive adhesive backed pads which are designed to be fastened to the patient adjacent to the wound site. Such pads may be configured with a means for clamping on the medical device to be secured. Such medical device anchoring systems are shown, by way of example, in U.S. Pat. Nos. 6,290,676 and 6,361,523. However, these anchoring systems may have drawbacks such as complexity of the system and use, inability to fully seal the wound site, and inability to prevent movement of the medical device in and out of the wound site.

Thus, there is a need in the art for a fixation system, device and method for rapidly and securely affixing a medical article to a patient that provides for greater patient comfort and other desirable characteristics.

### SUMMARY

Fixation systems, devices and methods meeting the needs in the art are provided. The fixation systems, devices and methods employ a base member in varying embodiments wherein the base member serves as a platform for securing an elongated medical article to the skin of a patient. The base member generally comprises an upper base member surface, a lower base member surface, an opening through the base member from the lower base member surface to the upper base member surface, and an arcuate channel projecting from the upper base member surface in proximity to the opening through the base member.

In an embodiment, a device for securing an elongated medical article to the skin of a patient is provided comprising a flexible member and a base member. The flexible member comprises a flexible material and a polymerization initiator or rate modifier permeated throughout at least a portion of said flexible material. The flexible member has a lower flexible member surface, an upper flexible member surface and an aperture through the flexible member. The base member comprises an upper base member surface, a lower base member surface affixed to the upper flexible member surface, an opening through the base member from the lower base member surface to the upper base member surface corresponding to the aperture in the flexible member, fluid communication between the upper base member surface and the lower base member surface, and an arcuate channel projecting from the upper base member surface in proximity to the opening through the base member.

In an embodiment, a fixation system for securing an elongated medical article to the skin of a patient is provided comprising a flexible member, a base member and an attachment member. The flexible member comprises a flexible material and a polymerization initiator or rate modifier permeated throughout at least a portion of the flexible material. The flexible member has a lower flexible member surface, an upper flexible member surface and an aperture through the flexible member. The base member comprises an upper base member surface, a lower base member surface affixed to the upper flexible member surface, an opening through the base member from the lower base member surface to the upper base member surface corresponding to the aperture in the flexible member, and an arcuate channel projecting from the upper base member surface in proximity to the opening through the base member. The attachment member is for attaching an elongated medical article to the arcuate channel on the base member. The fixation system may be provided in a kit. The fixation system in a kit may include an effective amount of polymerizable adhesive composition.

In one embodiment, a device for securing an elongated medical article to the skin of a patient is provided comprising a base member comprising an upper base member surface, a lower base member surface, an opening through the base member from the lower base member surface to the upper base member surface, fluid communication between the upper base member surface and the lower base member surface, an arcuate channel projecting from the upper base member surface in proximity to the opening through the base member, and a plurality of protrusions projecting from the lower base member surface. A wall portion may extend outwardly from and at least partially surround the perimeter of the lower base member surface.

In embodiments, the device may further comprise a passageway radially extending through the base member to the perimeter of the base member. In other embodiments, the device further comprises a recessed cavity in the lower base member surface around the opening through the base member. The recessed cavity may be extended to the perimeter of the base member. An absorptive foam member may be secured in the recessed cavity.

In another embodiment, a fixation system for securing an elongated medical article to the skin of a patient is provided comprising a base member comprising an upper base member surface, a lower base member surface having a plurality of protrusions integral with and projecting from the lower base member surface which contact the skin of the patient forming a gap between the lower base member surface and the skin of the patient, an opening through the base member from the lower base member surface to the upper base member surface for receiving the elongated medical article, fluid communication between the upper base member surface and the lower base member surface for application of a polymerizable adhesive composition to the gap between the lower base member surface and the skin of the patient, and an arcuate channel projecting from the upper base member surface in proximity to the opening through the base member; an amount of polymerizable adhesive composition effective to affix the base member to the skin; and an attachment member for attaching the elongated medical article to the arcuate channel on the base member. The base member may further comprise a wall portion extending outwardly from and at least partially surrounding the perimeter of the lower base member surface. The fixation system may be provided in a kit.

### BRIEF DESCRIPTION OF THE DRAWINGS

FIG. 1 is a perspective view of a fixation device in accordance with an embodiment.

FIG. 1a is a perspective view of a fixation device in accordance with the embodiment of FIG. 1 having a passageway for receiving a medical article.

FIG. 1b is a perspective view of a flexible member in accordance with an embodiment of FIG. 1.

FIG. 1c is a perspective view of an alternate flexible member in accordance with an embodiment of FIG. 1a.

FIG. 2 is a side view of the fixation device of FIG. 1 as affixed to the skin of a patient.

FIG. 3 is a perspective view of a fixation device in accordance with an embodiment.

FIG. 4 is a perspective view of the embodiment of FIG. 3 after the fixation device has received an elongated medical article.

FIG. 5 is a perspective view of a fixation device in accordance with an embodiment having a passageway for receiving a medical article.

FIG. 6 is a perspective view of the underside of a fixation device in accordance with an embodiment.

FIG. 6a is a perspective view of the underside of a fixation device illustrating one type of slot under the arcuate channel for an attachment member.

FIG. 7 is an exploded perspective view of the underside of an embodiment of a fixation device including an absorptive foam member.

FIG. 8 is a perspective view of the underside of the fixation device of FIG. 7 with the absorptive foam member and an elongated medical article positioned within the fixation device.

FIG. 9 is an exploded perspective view of the underside of an embodiment of a fixation device including an absorptive foam member with a pull tab.

FIG. 10 is a perspective view of the underside of the fixation device of FIG. 9 with the absorptive foam member and elongated medical article positioned within the fixation device.

### DETAILED DESCRIPTION

Fixation devices, systems and methods for rapidly and securely affixing a medical article to a patient are described that provide for greater patient comfort and provide a waterproof, microbial barrier to the medical article exit wound site. The fixation devices, systems and methods described substantially provide for the immobilization of a platform to a patient's skin for purposes of securing a medical article, particularly an elongated medical article. The medical article may be any type of medical article that may need to be secured to a patient for a period of time. The fixation systems, fixation devices and fixation methods provide means for distributing the strain-relieving load over a relatively broad area, leading to greater patient comfort. The patient may be human or non-human, typically a mammal.

“Affix” and grammatical equivalents thereof as used herein refers to any method of attachment, by way of example, but not limited to, adhering, connecting, bonding, fastening, joining, linking, coupling, or restraining a first part to a second part.

The fixation devices, systems and methods for affixing a medical article to the skin of a patient may include securing any elongated medical article which is or may be used percutaneously such that fixation to the skin of a patient is desirable. As used herein, “elongated medical article” includes, but is not limited to, medical devices, medical components or medical elements which have a length longer than the width or circumference of the device, component or element. By way of example, the elongated medical article may be a flexible percutaneous component, including but not limited to, fluid drainage articles such as surgical drainage tubes, feeding tubes, chest tubes, or nasogastric tubes, catheters such as IV catheters, arterial catheters, epidural catheters, mid-line catheters, central venous catheters, peripherally inserted central catheters, or hemodialysis catheters, or electrical wires or cables connected to external or implanted electronic devices or sensors such as temporary pacing leads. As used herein, the term “catheter” refers to all types of hollow tubular units employed either for removal of bodily fluids or for introduction of fluids into the body.

The fixation devices provided include a base member which functions as a platform for securing an elongated medical article. The base member generally comprises an upper base member surface, a lower base member surface, an opening through the base member from the lower base member surface to the upper base member surface for receiving the elongated medical article, and an arcuate channel projecting from the upper base member surface in proximity to the opening through the base member. Specific embodiments of the base member are described.

The arcuate channel projects from the upper base member surface in proximity to the opening through the base member. Thus, when a medical article is received in the fixation device, the arcuate channel is at or near the opening in the base member to aid in the securement of the medical article.

The arcuate channel may be proximally integral with the upper base member surface and extend distally from the opening in the upper base member surface. In embodiments, the arcuate channel extends from the opening in the upper base member surface, which opening is preferably in or near the center of the base member, providing a substantially centrally located opening.

The arcuate channel has a groove portion which is configured to receive at least a portion of the elongated medical article when the article is placed in or proximal to the groove in the arcuate channel. An attachment member may be used to hold the article to the groove in the arcuate channel.

An attachment member provides a strain relief element of the system and secures the medical device exiting the patient to the fixation device such that loads applied to the medical article by tugging or bumping are borne by the fixation device, and, thus, do not cause movement or dislodgement of the medical article from the wound site. The attachment member may be of any type of attachment such as a suture, a medical adhesive, a clamp, a clip, mechanical tape, a hook and loop fastener, an elastic band, a buckle, or other attachment or fastener device. The arcuate channel on the base member may have a slot therethrough for placing an attachment member around the arcuate channel holding the medical article and the medical article, thus securing the medical article at least partially in the groove of the arcuate channel. The slot may be formed in any desirable shape or size, preferably in the area under the groove in the arcuate channel projecting above the base member surface.

The opening in the base member may be any size or shape desirable for fixation of the medical article for which the fixation device is to be used. In the alternative, the opening may be a standard size which will work with medical articles of a variety of sizes. Thus, the fixation device may work with a variety of medical articles without having to have a different device for every size of medical article. The advantages of the use of the fixation device structure with the polymerizable adhesive composition allows for the use of the device with a variety of sizes of medical articles, while still providing the desired fixation or securement of the medical article through the features of the device. In one embodiment, the opening in the base member completely surrounds the medical article during use.

In one embodiment, a passageway may extend radially from the opening of the base member to the outer perimeter of the base member. The medical article may be received or removed from the base member by sliding or slipping the medical article through the passageway from the opening in the base member to the perimeter of the base member. The passageway from the opening in the base member to the perimeter of the base member may be sized to accommodate the outer diameter of the medical article. Alternatively, the medical article may be flexible such that the medical article may be squeezed prior to sliding the medical article through the passageway and in or out of the base member. In other embodiments, the material of the base member may be sufficiently flexible such that the passageway may be enlarged upon desiring to receive or remove the medical article through the passageway from the fixation device.

The base member may be any size desirable for the medical article which is to be fixed into place thereby. The base member may be any shape desired, such as square, rectangular, elliptical, oblong, among others, but will preferably be elliptical or circular, such as a circular or elliptical disc.

The base member includes fluid communication between the upper base member surface and the lower base member surface. In embodiments, the opening in the base member may be used as fluid communication provided the medical article and/or base member are flexible enough to permit such use. In other embodiments, the fluid communication may comprise one or more polymerizable adhesive composition application holes such as for application of polymerizable adhesive therethrough. The one or more holes may be provided in a random configuration or in a pattern on the base member. Preferably, the holes are uniformly distributed over the area of the base member such that the polymerizable adhesive composition may be added uniformly during use. Alternatively, the fluid communication may include a distribution member for distributing the polymerizable adhesive composition to the lower base member surfaces.

In one embodiment, a fixation device is provided which comprises a base member comprising an upper base member surface, a lower base member surface, an opening through the base member from the lower base member surface to the upper base member surface for receiving an elongated medical article, and an arcuate channel projecting from the upper base member surface in proximity to the opening through the base member. The base member further has a plurality of protrusions integral with and projecting outwardly from the lower base member surface. In embodiments, the protrusions project away from the lower base member. In embodiments, the protrusions independently are between about 0.010 to about 0.020 inch in length. The protrusions may be the same length or may be of varying lengths for purposes of particular needs for bonding with the surface area of the skin.

In embodiments where the base member comprises protrusions, the base member may further comprise a wall portion at least partially surrounding the perimeter of the base member and extending outwardly from the lower base member surface. The wall portion may be integrally formed with the base member. In embodiments, the wall portion extends substantially outwardly and substantially perpendicularly from the lower base member surface. The wall portion typically extends substantially the same distance from the lower base member surface as the protrusions. In embodiments, the wall portion may extend a greater distance from the lower base member surface than the protrusions.

In use, the fixation device receives the medical article and is positioned against the skin such that the wall portion and the protrusions contact the skin of the patient forming a gap between the lower base member surface and the skin of the patient. Typically, the protrusions provide a sufficient gap between the skin and the base member to allow for an effective amount of polymerizable adhesive composition to be placed in the gap. The polymerizable adhesive composition then polymerizes or is polymerized such that a bond between the fixation device and the skin is formed. An effective amount of polymerizable adhesive composition is the amount required to form a bond between the fixation device and the skin.

Upon application of a polymerizable adhesive composition, the length of the protrusions and wall portion define and control the thickness of the polymerizable material, and, ultimately, the polymerized adhesive formed during use beneath the fixation device, ensuring secure attachment. The polymerizable adhesive composition may be added through the hole in the base member or through other methods of fluid communication through the fixation device. The effective amount of polymerizable adhesive composition may depend on the type of elongated medical article being secured and can be determined for a particular use.

In embodiments, the fixation device having a plurality of protrusions projecting from the lower base member surface further includes fluid communication between the upper base member surface and the lower base member surface. In embodiments, the fluid communication comprises one or more polymerizable adhesive composition application holes from the upper base member surface through the lower base member surface such that a polymerizable adhesive composition may be applied or inserted through the holes into the gap between the lower base member surface and the skin of the patient. Upon application of the polymerizable adhesive composition, the wall portion surrounding the lower base member surface provides for containment of the polymerizable adhesive composition under the base member of the fixation device. The polymerizable adhesive composition, once applied to the gap formed between the base member and the skin, contacts the skin and polymerizes to form a seal around the medical article and bonds the fixation device to the skin.

Once the polymerizable adhesive composition is polymerized, an attachment member may be used for attaching the elongated medical article to the arcuate channel on the base member.

A fixation system is provided which includes (a) a base member comprising an upper base member surface, a lower base member surface, an opening through the base member from the lower base member surface to the upper base member surface for receiving an elongated medical article, a plurality of protrusions integral with and projecting from the lower base member surface, and an arcuate channel projecting from the upper base member surface in proximity to the opening through the base member; (b) an attachment member; and (c) an effective amount of a polymerizable adhesive composition. The base member may further comprise a wall portion surrounding at least a portion of the perimeter of the lower base member surface extending outwardly from the lower base member surface. The fixation system may be provided in a kit which may further comprise one or more additional elements useful in medical article fixation. By way of example, one or more medical articles may be included in the kit.

In another embodiment of the fixation device comprising a base member having protrusions extending therefrom, a recessed cavity is formed in the base member in the lower base member surface. Preferably, the recessed cavity is formed in a substantially symmetrical shape around the opening in the base member. The recessed cavity in the base member may be formed in a shape and size suitable for containing an absorptive foam member and may have a lip extending around the recessed cavity and outwardly from the lower base member surface. The lip around the recessed cavity prevents the polymerizable adhesive composition from flowing underneath the absorptive foam member in use. In embodiments, the recessed cavity is extended to the perimeter of the base member.

The extension of the recessed cavity may be used for sliding the absorptive foam member under the base member. By way of example, the absorptive foam member may include at least one pull tab to facilitate positioning the absorptive foam member in the recessed cavity. The extended recessed cavity may facilitate the placement and removal of the absorptive foam member as required for particular use, including providing the ability to place, remove, or replace the absorptive foam member while the fixation device is affixed/bonded to the patient's skin. The extension of the recessed cavity may be more shallow than the recessed cavity itself.

By way of example, the absorptive foam member may have a pull tab which may in embodiments be bifurcated with perforations to allow installing the absorptive foam member under the fixation device. The pull tab may be adhered to the top of the absorptive foam member with pressure sensitive adhesive, for example. The fixation device may be installed over the medical article, such as a surgical drain, and bonded to the patient with a polymerizable adhesive composition such as a cyanoacrylate monomer composition. The fixation device is positioned next to the patient's skin and the adhesive applied to the device to bond the device to the skin. The absorptive foam member is then installed by sliding the pull tab under the fixation device through the recessed cavity and extended recessed cavity. The pull tab may feed out the opposite side of the fixation device as the absorptive foam member slides under the fixation device into the recessed cavity. Once the absorptive foam member is fully positioned within the recessed cavity, the pull tab may be snapped off at the perforation, if any.

The absorptive foam member in embodiments comprises a first surface and a second surface and a radial slit extending from the edge of the foam member to a central point proximate to the center of the foam member. In use, the central point typically will correspond to the opening in the base member. The absorptive foam member may contain an antimicrobial agent. By way of example, suitable absorptive foam members are described in U.S. Pat. No. 5,833,665, incorporated herein by reference in its entirety. By further way of example, the absorptive foam member may be a BioPatch™ antimicrobial dressing.

In embodiments, the absorptive foam member comprises an elastomeric pad having a radial slit extending from the edge of the pad to a central point proximate to the center of the pad. The pad may comprise a cross-linked biopolymer and a bioactive reagent reversibly bound thereto, wherein the bioactive reagent is releasable from the cross-linked biopolymer in a controlled manner to the skin. The absorptive foam member may further comprise a reinforced, flexible, water vapor permeable membrane adhesively attached to the pad.

In embodiments, the absorptive foam member comprises a hydrophilic polyurethane absorptive foam and an antimicrobial agent. By way of example, the antimicrobial agent may be chlorhexidine gluconate.

The absorptive foam member may be placed around the medical article by means of the radial slit. In embodiments, the fixation device includes a recessed cavity in the base member, particularly in the lower base member surface, for placement of the absorptive foam member. The absorptive foam member may be affixed within the recessed cavity by pressure sensitive adhesive. Alternatively, the foam member may be captured within the recessed cavity between the base member and the patient skin when the device is applied to the patient. In embodiments, the base member may also include a passageway from the opening through the base member extending radially to the outer perimeter of the base member. The slit in the absorptive foam member and the passageway in the base member may be correspondingly aligned such that the medical article may be cooperatively received through the slit of the absorptive foam member and the passageway of the base member. One or more absorptive foam members may be included in a fixation system kit.

The recessed cavity formed or molded on the underside of the base member for the absorptive foam member has a lip around the recessed cavity in order to prevent the polymerizable adhesive composition upon application through the opening in the base member or through fluid communication such as polymerizable adhesive composition holes from flowing underneath the absorptive foam member. The lip typically extends around the recessed cavity and outwardly from the lower base member surface to about the same distance as the protrusions and/or the wall portion, thus forming an area under the base member for the polymerizable adhesive composition between the wall portion and the lip. By way of the wall portion and the lip around the recessed cavity for the absorptive foam member, the adhesive is contained under the base member and prevented from flowing under the absorptive foam member when the fixation device is placed on the skin or tissue to which it is to be affixed and the polymerizable adhesive composition is applied. When polymerization occurs, the fixation device containing the absorptive foam member is affixed to the skin.

The fixation device in this embodiment comprises a base member comprising an upper base member surface; a lower base member surface; an opening through the base member from the lower base member surface to the upper base member surface for receiving an elongated medical article; a plurality of protrusions integral with and projecting from the lower base member surface; a wall portion surrounding at least a portion of the perimeter of and extending outwardly from the lower base member surface; a recessed cavity in the base member in the lower base member surface; and an arcuate channel projecting from the upper base member surface in proximity to the opening through the base member. There may additionally be fluid communication between the upper base member and the lower base member. By way of example, the base member may further comprise one or more polymerizable adhesive composition application holes from the upper base member surface through the lower base member surface. A lip preferably surrounds the recessed cavity.

In embodiments wherein the base member comprises an upper base member surface, a lower base member surface, an opening through the base member from the lower base member surface to the upper base member surface for receiving an elongated medical article, and an arcuate channel projecting from the upper base member surface in proximity to the opening through the base member, the base member and arcuate channel may be formed or molded in one piece from a plastic material such as a thermoplastic elastomer material. Other materials may be used to adjust the properties of the base member for a particular purpose or use, by way of example, more flexible materials may be used depending on the medical article and the requirements of the base member in receiving the medical article. Alternatively, the arcuate channel may be formed separately and attached to the base member for use in securing an elongated medical article. The arcuate channel portion of the base member, if formed separately, may be used to control the size of the opening in the base member to accommodate varying sizes of medical articles.

The protrusions and wall portion projecting from the base member in one embodiment may be part of the molded piece. The recessed cavity and lip encircling the cavity may be a molded cavity on the underside or lower base member surface of the fixation device.

In one embodiment, the base member of the fixation device comprises an upper base member surface, a lower base member surface, an opening through the base member from the lower base member surface to the upper base member surface for receiving an elongated medical article, and an arcuate channel projecting from the upper base member surface in proximity to the opening through the base member. The lower base member in this embodiment is substantially planar and is affixed to a flexible member which may then be bonded to the skin of a patient. The flexible member has an upper flexible member surface, a lower flexible member surface and an aperture through the flexible member. The aperture in the flexible member corresponds to the opening in the base member for passage of an elongated medical article therethrough. The medical article may be received through the aperture in the flexible member and the opening in the base member which are lined up in corresponding position. The flexible member may extend beyond the perimeter of the base member.

In this embodiment, the flexible member with the base member adhered thereto is placed on the skin of the patient, an elongated medical article is received therethrough, and a polymerizable adhesive composition is applied to the flexible member and allowed to polymerize. Alternatively, the elongated medical article may be received through the fixation device, the fixation device comprising a flexible member adhered to a base member may be placed on the skin of the patient, and a polymerizable adhesive composition is applied to the flexible member and allowed to polymerize. The fixation device including a flexible member and base member, thus, is bonded to the skin by the polymerized adhesive. Once the polymerizable adhesive composition is polymerized, an attachment member may be used for attaching the elongated medical article to the arcuate channel on the base member.

The lower base member surface may be affixed to the upper flexible member surface by any means known to those of skill in the art. Typically, the base member may be affixed to the flexible member by use of a medically acceptable adhesive such as a medically acceptable pressure sensitive adhesive. The base member and flexible member preferably may be affixed to each other prior to use as a fixation device. In the alternative, the flexible member and then the base member may be placed separately over the medical article to be secured at the time of attachment of the medical article to the skin of the patient.

In one embodiment including a base member and a flexible member, the base member includes fluid communication between the upper base member surface and the lower base member surface. The fluid communication may include one or more polymerizable adhesive composition application holes from the upper base member surface through the lower base member surface. The holes provide access through the base member to the flexible member for application of a polymerizable adhesive composition upon positioning the fixation device on the patient's skin.

In an alternative embodiment, a polymerizable adhesive composition may be added along the edge of the fixation device, particularly along the edge of the flexible member.

In one embodiment, the fixation system primarily comprises a flexible member and a base member which comprise a fixation device. An attachment member may be used with the fixation system. The flexible member is provided with an effective amount of polymerizable adhesive composition. The fixation system may be included in a kit which may include an attachment member, additional polymerizable adhesive composition and/or one or more medical articles, for example.

The flexible member and base member provide a structure to affix the medical article to a patient's skin. The polymerizable adhesive composition affixes the fixation device to the patient upon polymerization of the adhesive composition and provides a waterproof/microbial barrier to the wound site.

The flexible member may comprise a flexible material and a polymerization initiator or rate modifier. The polymerizable adhesive composition may be a polymerizable monomer composition such as a polymerizable cyanoacrylate composition wherein the polymerizable monomer composition is applied to the flexible member of the fixation system after the fixation system is positioned on the skin and allowed to polymerize, thus bonding the flexible member to the skin.

The flexible member may comprise a flexible material such as a mesh. The mesh may be configured with an aperture in the flexible material to allow passage of the medical article. As used herein, the term “aperture” means any hole, gap, or slit in the flexible material. The aperture may be positioned in the flexible material to provide uniform distribution of polymerizable adhesive material around the medical article, which may increase the stabilization of the medical article. The flexible material may be configured such that the outer perimeter of the flexible material includes a slit corresponding to a passageway in the base member, which slit allows the flexible material to receive the medical device, which is typically already positioned in a patient's body, or the flexible material may be continuous around the aperture such that the medical article is placed through the aperture upon use.

The flexible member has an upper surface and a lower surface. The lower surface typically is in contact with and will become adhered to the patient's skin or tissue. The upper surface typically is adhered to the base member.

The size and geometry of the flexible member may be varied depending on the nature of the medical article to be fixed or secured and the specific site on the patient where the article is to be placed. By way of example, a large drain catheter on an abdomen would require a different shape/size mesh than a small IV catheter for the back of the hand.

In embodiments, the flexible member may be provided coated with a layer of pressure sensitive adhesive (PSA) on at least a portion of one side to facilitate temporary fixation of the flexible material to the patient's skin in the desired location prior to placement of the polymerizable adhesive composition. The flexible member may further be coated or impregnated with a chemical initiator to control the polymerization of the polymerizable adhesive composition applied later to fully secure the fixation device in place.

The flexible member provides visualization of the medical article site, a suitably robust mechanical lattice to secure the base member and connecting member to, and flexibility to conform to the topography of the patient. Additionally, the pores in the flexible member allow the polymerizable adhesive to penetrate through to the surface of the skin, providing a continuous bond across a portion of or the entire surface of the flexible member which is in contact with the patient's skin.

In embodiments, the flexible member comprises flexible material which will be a material which may be applied to a surface, and impregnated with a polymerizable monomeric adhesive composition, which upon setting or curing provides an adherent structure over the surface. Polymerization (setting or curing) of the polymerizable monomeric adhesive composition is assisted by the flexible material being loaded, coated, or the like with a polymerization initiator or rate modifier for the polymerizable monomeric adhesive composition.

In embodiments, the flexible material can be formed of any suitable flexible or compliant material suitable for use in a fixation device. Preferably, the flexible material is a material that is flexible, porous, and non-toxic. As used herein, the term “flexible” is used to refer to the flexible material. However, unless stated differently in context, the term “flexible” is meant to cover a range of materials, which exhibit one or more properties such as being flexible, compliant, elastic, or memory retentive. For example, “flexible” is also meant to refer to materials that exhibit elastic or memory properties, i.e., the ability for the material to return to its original shape when stresses applied thereto are reduced or eliminated.

The flexible material is preferably flexible or compliant, to allow the flexible substrate to be placed on the desired surface (such as skin, organ, tissue, or the like) in a manner that allows the flexible substrate to conform to the topology of the desired surface. Likewise, the flexible material is preferably porous, to allow the subsequently applied polymerizable adhesive material to pass through or permeate through the flexible material and to polymerize as a layer beneath the flexible material, while adhering the flexible material to the desired substrate. By “porous” is meant herein either that the bulk of the flexible material has pores, such that the applied polymerizable adhesive composition is soaked up or absorbed by the bulk material, or that the bulk of the flexible material has voids (like a net or screen), such that the applied polymerizable adhesive composition passes directly through the bulk material, with or without being soaked up or absorbed by the bulk material. For example, in the case of textile materials, “porous” is generally used to mean that the applied polymerizable adhesive composition permeates and passes through interstices between the fibers, but does not necessarily pass into and through the fibers themselves.

Such porosity (or other properties such as hydrophobicity or hydrophilicity) will also allow a polymerization initiator or rate modifier to be loaded on the flexible material prior to use, to initiate a subsequently applied polymerizable adhesive material. The flexible material is also preferably non-toxic, as it is intended to be used as on biological tissues. As such, the flexible material should be biologically compatible with the desired substrate (such as tissue, skin, organ, or the like), and is preferably a material that is governmentally approved or generally regarded as safe for the desired purpose.

In other embodiments, the flexible material may be selected to be elastic or have some memory effect. In such embodiments, the elastic properties of the flexible material may desirably provide a degree of pressure or stress at the application site, for example, to maintain a sealing effect around or on the damaged or wounded tissue through which the medical article was placed. Likewise, in embodiments where such additional degree of pressure or stress at the application site is not desired, the flexible material may be selected to have less or no elasticity.

In embodiments, it is preferred that the flexible material is a textile or mesh/web material. Suitable textile materials can be formed of either synthetic or natural materials. Such textile material can be formed of either woven or non-woven fabrics or materials. The flexible material may be, for example, any suitable polymeric film, plastic foam (including open celled foam), a woven fabric, knitted fabric, a non-woven fabric, mixture thereof, or the like. In particular, suitable flexible materials may thus be prepared, for example, from nylon, a polyolefin film, such as polyethylene, polypropylene, ethylene propylene copolymers, and ethylene butylene copolymers, polyurethanes, polyurethane foams, polystyrenes, plasticized polyvinylchlorides, polyesters, polyamides, and cotton. Suitable specific examples include, for example, nylon, polyethylene, polypropylene, ethylene propylene copolymers, ethylene butylene copolymers, polyurethane, polystyrene, plasticized polyvinylchloride, polyester, polyamide, cotton, polytetrafluoroethylene (PTFE), biovascular material, collagen, Gore-Tex®, AND DACRON®, among others.

In some embodiments, it is preferred that the textile material not be formed of elastin, or elastin-based materials. Although elastin may be suitable for some uses, synthetic materials are preferred in embodiments in view of their availability, ease of manufacture, physical properties such as strength and durability, and biological compatibility. Thus, in such embodiments, it is preferred that the textile material is substantially or completely free of elastin or elastin-based materials. Further, in such embodiments, it is preferred that the entire flexible member (i.e., the combination of the flexible material and the adhesive substance) is substantially or completely free of elastin or elastin-based materials.

In embodiments, the flexible material may be formed of a synthetic, semi-synthetic, or natural organic material. Thus, for example, the flexible material may be formed of a synthetic or natural polymer material, but not from a material such as metal (such as silver, steel or the like) or glass or ceramic.

The flexible material is preferably resistant to tearing. In one embodiment, the thickness of the flexible material of the present invention is from about 1 mil to about 50 mils. In another embodiment, the thickness of the flexible material is from about 1 mil to about 20 mils, preferably from about 1 mil to about 10 mils, or from about 1 mil to about 5 mils.

The flexible material may be opaque or translucent. In some embodiments of the present invention, the flexible material is provided to have a skin color, such that the flexible material masks the appearance of the underlying surface (such as a wound). However, in other embodiments, the flexible material can be provided with “designer” colors and/or patterns, or even cartoon character designs. In other embodiments, the flexible material may be clear, thus not masking the underlying surface through which the medical article passes.

The flexible member may be of any configuration found useful to those skilled in the art. For example, although shown in the Figures as substantially circular, the flexible member may be in rectangular or square configurations, or the flexible member can take any number of other shapes, which can be designed for particular applications. For example, circular or round flexible materials typically are used in view of the shape and sizes of the typically used medical articles. However, the flexible member may be in other shapes, such as oval, elliptical, triangular, polygonal, semi-circular, and the like, in embodiments.

In some embodiments, the flexible member can include a pressure sensitive adhesive on at least one face, to assist in initial placement of the flexible member on the desired surface.

In embodiments where the flexible material includes a pressure sensitive adhesive applied to portions of the flexible material, the pressure sensitive adhesive can be applied to an entire surface of the flexible material, or only to portions (such as peripheral edges) of the surface of the flexible material. The exposed pressure sensitive adhesive can be covered by a suitable release layer or liner, if desired, to preserve the adhesiveness of the flexible material until time of use. The pressure sensitive adhesive, if present, can be applied in the various manners shown in U.S. Patent Application Publication No. 2005/0182443, the entire disclosure of which is incorporated herein by reference.

In embodiments wherein an adhesive substance such as a pressure sensitive adhesive is used, the flexible member preferably includes an adhesive substance applied only to portions of the flexible material. Preferably, the adhesive substance is applied to the flexible material on opposite sides or ends of the flexible material. In this manner, the flexible member can be applied over skin punctured by a percutaneous medical article such that the portion of the flexible member not coated with the adhesive substance straddles the area surrounding the medical article. Accordingly, the adhesive substance is applied to the lower flexible member surface of the flexible member, and the exposed adhesive substance can be covered by a suitable release layer or liner to preserve the adhesiveness of the flexible member until time of use.

In another embodiment, the flexible member can be coated on the lower flexible member surface with an adhesive substance. In this embodiment, the adhesive substance can be located on substantially an entire surface of the flexible substrate, rather than only on opposing edges of the flexible substrate as described above. When prepared in this manner, the adhesive substance can be coated to cover the entire surface in a continuous coating or layer. Alternatively, or preferably in some embodiments, the coating is discontinuous to provide areas that are not covered by the adhesive substance, such as by the adhesive substance being provided in a form of regular or random spots, lines, or the like. Where the adhesive substance does not cover the entire surface of the flexible substrate to form a continuous layer, it is preferred that the adhesive is coated on at least 25% but no more than 75% of the surface area, and more preferably between about 40 and about 60% of the surface area.

When the flexible member is provided according to this embodiment, it is preferred that the adhesive substance applied to the surface of the flexible member be a pressure sensitive adhesive, which preferably exhibits a low degree of adhesiveness. The adhesive substance to be applied can be, if desired, the same as the adhesive substance described above, which is applied to only portions of the flexible member. Or, the adhesive substance used in this embodiment can be a weaker or different adhesive substance. That is, the purpose of the adhesive substance is only to maintain the flexible member in position on the desired surface, and optionally provide a minimal adhesion force until the polymerizable cyanoacrylate adhesive composition is applied and allowed to set to fully adhere the flexible member to the desired surface. The adhesive substance is thus weak enough to allow the applied polymerizable adhesive material to penetrate through the flexible member and the applied adhesive substance, to form a polymerized bond between the flexible member (and applied adhesive substance) and the underlying desired substrate, such as the skin.

In this embodiment, any suitable adhesive substance can be used, as desired. Preferably, the adhesive substance should be non-toxic, and capable and/or approved for use on biological surfaces. Suitable adhesive substances thus include, for example, those adhesive substances commonly used in production of conventional adhesive bandages. Furthermore, in this embodiment where the adhesive substances covers substantially an entire face of the flexible material, and thus remains in the final composite structure, it is preferred that the polymerizable adhesive composition be able to interact with and/or solubilize the adhesive substances. That is, it is preferred that the polymerizable adhesive composition be able to in essence replace the adhesive substance as the primary means of attaching the composite structure to the underlying skin. This can occur, for example, either by the polymerizable adhesive composition solubilizing the adhesive substance, or by the polymerizable adhesive composition being able to bond the flexible member to the underlying substrate through gaps or voids either pre-existing or created in the adhesive substance layer.

A suitable backing or release material may be used to cover the adhesive substances applied to the lower flexible member surface. Such backing materials are well known in the art for covering pressure sensitive adhesives and can include, for example, paper, plastic, or the like.

The adhesive substance, when used, is typically present in coat weight from about 10 to about 200, or from about 20 to 150 grams per square meter (gsm). Of course, other coat weights of the adhesive substance can be used, as desired.

The adhesive substance used in the flexible member may, for example, be any suitable adhesive substance. Preferably, the adhesive substance is a medical grade adhesive, such as acrylic based pressure sensitive adhesives (PSAs), rubber based pressure sensitive adhesives, silicone pressure sensitive adhesives, mixtures thereof, or the like. In embodiments, it is preferred that the adhesive substance be different from the polymerizable adhesive composition. Thus, for example, it is preferred that while the polymerizable adhesive composition can be, for example, a polymerizable monomeric adhesive composition, the adhesive substance is an adhesive material that is not a polymerizable adhesive composition, such as a pressure sensitive adhesive.

Suitable rubber based PSAs include, but are not limited to, those taught in U.S. Pat. No. 5,705,551 and in U.S. Pat. No. 4,080,348, the disclosures of which are hereby incorporated by reference. Examples of polymeric rubber bases include one or more of styrene-isoprene-styrene polymers, styrene-olefin-styrene polymers including styrene-ethylene/propylene-styrene polymers, polyisobutylene, styrene-butadiene-styrene polymers, polyisoprene, polybutadiene, natural rubber, silicone rubber, acrylonitrile rubber, nitrile rubber, polyurethane rubber, polyisobutylene rubber, butyl rubber, halobutyl rubber including bromobutyl rubber, butadiene-acrylonitrile rubber, polychloroprene, and styrene-butadiene rubber.

A particularly useful rubber based adhesive is that which has a thermoplastic elastomeric component and a resin component. The thermoplastic elastomeric component contains about 55-85 parts of a simple A-B block copolymer wherein the A-blocks are derived from styrene homologs and the B-blocks are derived from isoprene, and about 15-45 parts of a linear or radical A-B-A block copolymer wherein the A-blocks are derived from styrene or styrene homologs and the B-blocks are derived from conjugated dienes or lower alkenes, the A-blocks in the A-B block copolymer constituting about 10-18 percent by weight of the A-B copolymer and the total A-B and A-B-A copolymers containing about 20 percent or less styrene. The resin component consists of essentially of tackifier resins for the elastomeric component. In general any compatible conventional tackifier resin or mixture of such resins may be used. These include hydrocarbon resins, rosin and rosin derivatives, polyterpenes and other tackifiers. The adhesive composition contains about 20-300 parts of the resin component per one hundred parts by weight of the thermoplastic elastomeric component. One such rubber based adhesive is commercially available from Ato Findley under the trade name HM3210.

Useful acrylic based PSAs include, but are not limited to, those taught in U.S. Pat. No. 5,947,917 and U.S. Pat. No. 5,164,444 (acrylic emulsion), U.S. Pat. No. 5,623,011 (tackified acrylic emulsion). It can also be radiation curable mixture of monomers with initiators and other ingredients such as those taught in U.S. Pat. No. 5,232,958 (UV cured acrylic) and U.S. Pat. No. 5,232,958 (EB cured). The disclosures of these patents are hereby incorporated by reference.

It is contemplated that any acrylic based polymer capable of forming an adhesive layer with sufficient tack to adhere to the flexible member, the release liner or to a substrate, and with acceptable adhesion to skin, may function in the present invention. In certain embodiments, the acrylic polymers for the pressure-sensitive adhesive layers include those formed from polymerization of at least one alkyl acrylate monomer or methacrylate, an unsaturated carboxylic acid and optionally a vinyl lactam. Examples of suitable alkyl acrylate or methacrylate esters include, but are not limited to, butyl acrylate, ethyl acrylate, 2-ethylhexyl acrylate, isooctyl acrylate, isononyl acrylate, isodecyl acrylate, methyl acrylate, methylbutyl acrylate, 4-methyl-2-pentyl acrylate, sec-butyl acrylate, ethyl methacrylate, isodecyl methacrylate, methyl methacrylate, and the like, and mixtures thereof. Examples of suitable ethylenically unsaturated carboxylic acids include, but are not limited to, acrylic acid, methacrylic acid, fumaric acid, itaconic acid, and the like, and mixtures thereof. A preferred ethylenically unsaturated carboxylic acid monomer is acrylic acid. Examples of suitable vinyl lactams include, but are not limited to, N-vinyl caprolactam, 1-vinyl-2-piperidone, 1-vinyl-5-methyl-2-pyrrolidone-, vinyl pyrrolidone, and the like, and mixtures thereof.

The adhesive substance may also include a tackifier. Tackifiers, are generally hydrocarbon resins, wood resins, rosins, rosin derivatives, and the like. It is contemplated that any tackifier known by those of skill in the art to be compatible with elastomeric polymer compositions may be used. One such tackifier, found to be useful is Wingtak 10, a synthetic polyterpene resin that is liquid at room temperature, and sold by the Goodyear Tire and Rubber Company of Akron, Ohio. Wingtak 95 is a synthetic tackifier resin also available from Goodyear that comprises predominantly a polymer derived from piperylene and isoprene. Other suitable tackifying additives may include Escorez 1310, an aliphatic hydrocarbon resin, and Escorez 2596, a C₅-C₉ (aromatic modified aliphatic) resin, both manufactured by Exxon of Irving, Tex. Of course, as can be appreciated by those of skill in the art, a variety of different tackifying additives may be used.

In addition to the tackifiers, other additions may be included in the adhesive substances to impart desired properties. For example, plasticizers may be included and they are known to decrease the glass transition temperature of an adhesive composition containing elastomeric polymers. Shellflex 371 plasticizer is an example of a useful naphthenic processing oil available from Shell Oil Company of Houston, Tex. Antioxidants also may be included on the adhesive substance. Also included as suitable are Irgafos 168 antioxidant and Irganox 565 antioxidant available from Ciba-Geigy, Hawthorne, N.Y. Cutting agents such as waxes and surfactants also may be included in the adhesive substance.

Other optional materials that may be added to the adhesive substance layer in minor amounts (typically less than about 25% by weight of the elastomeric phase) include pH controllers, medicaments, bactericides, growth factors, wound healing components such as collagen, antioxidants, deodorants, perfumes, antimicrobials and fungicides.

Useful silicone pressure sensitive adhesives include those commercially available from Dow Corning Corp., Medical Products and those available from General Electric. Examples of silicone adhesives available from Dow Corning include those sold under the trademarks BIO-PSA X7-3027, BIO-PSA X7-4919, BIO-PSA X7-2685, BIO-PSA X7-3122 and BIO-PSA X7-4502. Additional examples of silicone pressure sensitive adhesives are described in U.S. Pat. Nos. 4,591,622, 4,584,355, 4,585,836 and 4,655,767, the entire disclosures of which are incorporated herein by reference.

The methods described for securing an elongated medical article are with reference to closing and covering a site on a tissue surface through which a medical device is exiting the patient. However, the invention is not limited to this embodiment.

In one embodiment, a method for securing an elongated medical article to the skin of a patient includes providing a device for securing a percutaneously positioned elongated medical article to the skin of a patient, positioning the device in cooperative relationship with the elongated medical article and in proximity to the skin, applying a polymerizable adhesive composition to the fixation device, and allowing the polymerizable adhesive composition to polymerize, forming a composite structure bonded to the skin. After the polymerization is completed, the elongated medical article may be attached to the fixation device with an attachment member.

In a more specific embodiment, a method for securing an elongated medical article to the skin of a patient comprises providing a device for securing a percutaneously positioned elongated medical article to the skin of a patient. The device comprises a flexible member comprising a flexible material, the flexible member having a lower flexible member surface, an upper flexible member surface and an aperture through the flexible member; and a base member comprising an upper base member surface, a lower base member surface affixed to the upper flexible member surface, an opening through the base member from the lower base member surface to the upper base member surface corresponding to the aperture in the flexible member, and an arcuate channel projecting from the upper base member surface in proximity to the opening through the base member. The fixation device is positioned in cooperative relationship with the elongated medical article and in proximity to the skin such that the percutaneously positioned elongated medical article is received in the fixation device. At least a portion of the flexible member is contacted with a polymerizable adhesive composition such that the polymerizable adhesive composition permeates the flexible member and polymerizes, bonding the device to the skin. The percutaneously positioned elongated medical article is optionally attached to the arcuate channel.

Typically, the polymerizable adhesive composition is allowed to permeate into and under the flexible member and polymerize to form a composite structure bonded to the skin. After the polymerization is completed, the elongated medical article is attached to the arcuate channel of the base member using an attachment member.

In embodiments, the fixation device including the flexible member receives the medical device exiting the patient's body or desired surface. The lower flexible member surface is placed proximate to the skin. For ease of application, the skin surface upon which the fixation device is to be applied is preferably horizontal, to help avoid slipping of the flexible material from the application site prior to complete polymerization of the subsequently applied polymerizable adhesive composition. However, where horizontal application is not possible or practical, the flexible material can be held in place by any suitable means including, but not limited to, by hand, forceps, tape, pressure sensitive adhesive, pressure, vacuum, or the like.

In a modification of this application method, a portion of a polymerizable adhesive material is applied to the application site prior to applying the fixation device. When so applied, the polymerizable adhesive material may or may not be allowed to fully polymerize prior to application of the fixation device and subsequent application of further amounts of polymerizable adhesive material. The polymerizable adhesive material applied prior to application of the fixation device can be the same as or different from the polymerizable adhesive material subsequently applied to the flexible member of the fixation device.

The polymerizable adhesive composition, such as a polymerizable monomeric adhesive composition, is applied over at least a portion of the flexible member. Preferably, the polymerizable adhesive composition is applied to fully cover the flexible member. However, if desired, a lesser amount of the polymerizable adhesive composition can be used to conserve materials and assist in subsequent removal. For example, if a portion of the flexible member is not covered by the polymerizable adhesive composition, that portion may be used either to maintain control over the flexible member during placement and polymerization, and then subsequently trimmed off, or it can be maintained and used as a tab to assist in subsequent removal (such as by sloughing off or peeling off of the composite structure, or by the use of a remover substance). In this instance, the polymerizable adhesive composition is preferably applied to the flexible member at least in an area sufficient to cover the underlying site of insertion of the medical article.

In this step of applying the polymerizable adhesive composition, an effective amount of polymerizable adhesive composition should be applied to form the desired composite structure once the polymerizable adhesive composition has polymerized (or cured). Thus, for example, the amount of polymerizable adhesive composition should be sufficient to preferably allow the composition to permeate through the flexible member to form a continuous coating between the flexible member and skin, which continuous coating subsequently polymerizes or cures to form a continuous polymeric coating between the flexible member and the underlying skin surface. The quantity of polymerizable adhesive composition should preferably further allow for a quantity of the composition to remain in, and preferably over, the flexible member. This further amount of polymerizable adhesive composition polymerizes or cures with the remaining polymerizable adhesive composition to provide a unitary composite structure that is bonded to the underlying surface.

If necessary or desired, the step of applying polymerizable adhesive composition to the flexible member can be repeated one or more times.

The flexible member may be contacted with the polymerizable adhesive composition through the opening in the base member through which the medical article extends or by contacting any portion of the flexible member that may extend past the outer edge of the base member in the fixation device. The fixation device may be structured such that the flexible member extends past a portion of the perimeter of the base member or entirely past the perimeter of the base member. In such embodiments, the polymerizable adhesive material may be applied around the edge of the base member only or around the edge and through the opening in the base member.

In a further embodiment, the base member includes fluid communication between the upper base member surface and the lower base member surface. In embodiments, the fluid communication is achieved by one or more polymerizable adhesive composition application holes from the upper base member surface through the lower base member surface wherein the polymerizable adhesive composition is permeated through at least a portion of the flexible member by application of the polymerizable adhesive composition through the polymerizable adhesive composition application holes in the base member. The base member may include a plurality of polymerizable adhesive composition application holes. As used herein, “plurality” means at least two. The polymerizable adhesive composition application holes may be spaced evenly or symmetrically over the base member or may be placed in a concentric or other design. The polymerizable adhesive composition application holes may be any size desired for applying the polymerizable adhesive composition, but preferably are not so large as to affect the structural integrity of the base member. Moreover, the plurality of holes will not be such that the number of holes will affect the structural integrity of the base member.

Once the polymerizable adhesive composition is cured, it forms a composite structure with the flexible member and the base member, covering the desired surface. The composite structure is adherent to the underlying surface, and provides the benefits described.

In another embodiment, a method for securing an elongated medical article to the skin of a patient is provided utilizing a fixation device including a base member with a plurality of protrusions on the lower base member surface for contacting the skin of the patient. The method includes providing a device for securing a percutaneously positioned elongated medical article to the skin of a patient, the device comprising a base member comprising an upper base member surface, a lower base member surface having a plurality of protrusions integral with and projecting from the lower base member surface which contact the skin of the patient forming a gap between the lower base member surface and the skin of the patient, an opening through the base member from the lower base member surface to the upper base member surface for receiving the elongated medical article, one or more polymerizable adhesive composition application holes from the upper base member surface through the lower base member surface for application of a polymerizable adhesive composition to the gap between the lower base member surface and the skin of the patient, and an arcuate channel projecting from the upper base member surface in proximity to the opening through the base member. A wall portion may extend outwardly from the lower base member surface around the gap between the lower base member surface and the skin of the patient. An attachment member for attaching an elongated medical article to the arcuate channel on the base member may be provided.

In this embodiment, a medical article which has been placed percutaneously into the patient through the skin of the patient is received by the fixation device and the fixation device is positioned onto the skin. Some or all of the protrusions rest directly on the skin, providing a gap between the skin and the lower base member surface. Polymerizable adhesive is allowed to contact the skin through the holes in the base member and the polymerizable adhesive polymerizes, forming a bond with the skin between the skin and the lower base member surface of the fixation device. The polymerizable adhesive composition holes may be provided such that one or more holes is used for delivering the polymerizable adhesive composition and one or more other holes is available to vent air from under the fixation device. The wall portion contains the polymerizable adhesive composition. An attachment member for attaching the medical article to the arcuate channel on the base member may then be used.

In another embodiment, the base member includes a recessed cavity containing an absorptive foam member. The recessed cavity is in the lower base member surface and surrounds the opening in the base member. Thus, the opening through the base member extends from the upper base member surface through the lower base member surface which includes the recessed cavity for the absorptive foam member.

The use of a liquid polymerizable adhesive composition as described provides a seal substantially completely around the shaft of the medical device as it exits the body. Without being bound by any theory, it is believed that this seal will limit small in/out movement of the catheter that could convey microbes into the wound site leading to infection. Advantages of the fixation device include providing the seal which gives a waterproof, microbial barrier to the medical article exit wound site, and allows for good vacuum to be maintained when fluid is being drained from a wound to allow for effective tissue approximation within a wound.

Of course, other methods will be readily apparent to those skilled in the art. The application methods are in no way limited to the methods described above.

The polymerizable adhesive composition may include one or more polymerizable monomers, which preferably are synthetic or semi-synthetic monomers. Preferred monomers that may be used in this invention are readily polymerizable, e.g. anionically polymerizable or free radical polymerizable, or polymerizable by zwitterions or ion pairs to form polymers. Such monomers include those that form polymers, that may, but do not need to, biodegrade. Such monomers are disclosed in, for example, U.S. Pat. Nos. 5,328,687, 5,928,611, 6,183,593, and U.S. Patent Publication No. 2004/0137067, which are hereby incorporated in their entirety by reference herein.

Preferred monomers include 1,1-disubstituted ethylene monomers, such as α-cyanoacrylates including, but not limited to, alkyl α-cyanoacrylates having an alkyl chain length of from about 1 to about 20 carbon atoms or more, preferably from about 3 to about 8 carbon atoms.

The α-cyanoacrylates of the present invention can be prepared according to several methods known in the art. U.S. Pat. Nos. 2,721,858, 3,254,111, 3,995,641, and 4,364,876, each of which is hereby incorporated in its entirety by reference herein, disclose methods for preparing α-cyanoacrylates.

Preferred α-cyanoacrylate monomers used in this invention include methyl cyanoacrylate, ethyl cyanoacrylate, n-butyl cyanoacrylate, 2-octyl cyanoacrylate, methoxyethyl cyanoacrylate, ethoxyethyl cyanoacrylate, dodecyl cyanoacrylate, 2-ethylhexyl cyanoacrylate, butyl cyanoacrylate, 3-methoxybutyl cyanoacrylate, 2-butoxyethyl cyanoacrylate, 2-isopropoxyethyl cyanoacrylate, 1-methoxy-2-propyl cyanoacrylate, hexyl cyanoacrylate, or dodecylcyanoacrylate.

Other suitable cyanoacrylates for use in the present invention also include, but are not limited to, alkyl ester cyanoacrylate monomers such as those having the formula

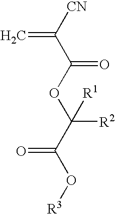

wherein R¹ and R² are, independently H, a straight, branched or cyclic alkyl, or are combined together in a cyclic alkyl group, and R³ is a straight, branched or cyclic alkyl group. Preferably, R¹ is H or a C₁, C₂ or C₃ alkyl group, such as methyl or ethyl; R² is H or a C₁, C₂ or C₃ alkyl group, such as methyl or ethyl; and R³ is a C₁-C₁₆ alkyl group, more preferably a C₁-C₁₀ alkyl group, such as methyl, ethyl, propyl, butyl, pentyl, hexyl, heptyl, octyl, nonyl or decyl, and even more preferably a C₂, C₃ or C₄ alkyl group. Such alkyl ester cyanoacrylates and other suitable monomers are disclosed in, for example, U.S. patent application publication 2002/0037310, and U.S. Pat. No. 6,620,846, the entire disclosures of which are incorporated herein by reference.

Examples of preferred alkyl ester cyanoacrylates include, but are not limited to, butyl lactoyl cyanoacrylate (BLCA), butyl glycoloyl cyanoacrylate (BGCA), ethyl lactoyl cyanoacrylate (ELCA), and ethyl glycoloyl cyanoacrylate (EGCA). BLCA may be represented by the above formula, wherein R¹ is H, R² is methyl and R³ is butyl. BGCA may be represented by the above formula, wherein R¹ is H, R² is H and R³ is butyl. ELCA may be represented by the above formula, wherein R¹ is H, R² is methyl and R³ is ethyl. EGCA may be represented by the above formula, wherein R¹ is H, R² is Hand R¹ R³ is ethyl.

The composition may optionally also include at least one plasticizing agent that assists in imparting flexibility to the polymer formed from the monomer. The plasticizing agent preferably contains little or no moisture and should not significantly affect the stability or polymerization of the monomer. Examples of suitable plasticizers include but are not limited to acetyl tributyl citrate, dimethyl sebacate, dibutyl sebacate, triethyl phosphate, tri(2-ethylhexyl)phosphate, tri(p-cresyl)phosphate, glyceryl triacetate, glyceryl tributyrate, diethyl sebacate, dioctyl adipate, isopropyl myristate, butyl stearate, lauric acid, trioctyl trimellitate, dioctyl glutarate, polydimethylsiloxane, and mixtures thereof. In embodiments, plasticizers may include tributyl citrate, acetyl tributyl citrate or dibutyl sebacate. In embodiments, suitable plasticizers include polymeric plasticizers, such as polyethylene glycol (PEG) esters and capped PEG esters or ethers, polyester glutarates and polyester adipates.

The composition may also optionally include at least one thixotropic agent. Suitable thixotropic agents are known to the skilled artisan and include, but are not limited to, silica gels such as those treated with a silyl isocyanate, and optionally surface treated titanium dioxide. Examples of suitable thixotropic agents and thickeners are disclosed in, for example, U.S. Pat. No. 4,720,513, and U.S. Pat. No. 6,310,166, the disclosures of which are hereby incorporated in their entireties by reference herein.

The composition may optionally also include thickeners. Suitable thickeners may include poly(2-ethylhexy methacrylate), poly(2-ethylhexyl acrylate) and others as listed in U.S. Pat. No. 6,183,593, the disclosure of which is incorporated by reference herein in its entirety.

The composition may also optionally include at least one natural or synthetic rubber to impart impact resistance. Suitable rubbers are known to the skilled artisan. Such rubbers include, but are not limited to, dienes, styrenes, acrylonitriles, and mixtures thereof. Examples of suitable rubbers are disclosed in, for example, U.S. Pat. Nos. 4,313,865 and 4,560,723, the disclosures of which are hereby incorporated in their entireties by reference herein.

The composition may optionally also include one or more stabilizers, preferably both at least one anionic vapor phase stabilizer and at least one anionic liquid phase stabilizer. These stabilizing agents may inhibit premature polymerization. Suitable stabilizers may include those listed in U.S. Pat. No. 6,183,593, the disclosure of which is incorporated by reference herein in its entirety. Furthermore, certain stabilizers may also function as anti-microbial agents, such as, for example, various acidic anti-microbials, as identified above.

The compositions may also include pH modifiers to control the rate of degradation of the resulting polymer, as disclosed in U.S. Pat. No. 6,143,352, the entire disclosure of which is hereby incorporated by reference herein in its entirety.

To improve the cohesive strength of adhesives formed from the compositions of this invention, difunctional monomeric cross-linking agents may be added to the monomer compositions of this invention. Such crosslinking agents are known. U.S. Pat. No. 3,940,362 to Overhults, which is hereby incorporated herein in its entirety by reference, discloses exemplary cross-linking agents.

The compositions of this invention may further contain colorants such as dyes, pigments, and pigment dyes.

The polymerizable compositions useful in the present invention may also further contain one or more preservatives, for prolonging the storage life of the composition. Suitable preservatives, and methods for selecting them and incorporating them into adhesive compositions, are disclosed in U.S. Pat. No. 6,579,469, the entire disclosure of which is incorporated herein by reference. Such preservatives can be in addition to any anti-microbial agent that may or may not be added to the composition. Such preservatives can be included irrespective of whether the composition and containers are sterilized.

When used, the flexible material preferably includes one or more chemical materials located within the flexible material. For example, one or more chemical substances can be dispersed in the flexible material, such as being chemically bound, physically bound, absorbed, or adsorbed to the flexible material. Thus, for example, the flexible material includes at least a polymerization initiator or rate modifier, and can optionally include one or more bioactive materials. As desired, the one or more chemical substances can be either immobilized on the flexible material, for example so that it has a desired effect but is not detached from the flexible material during use, or it can be attached to the flexible material in a manner such that it becomes detached during use.

For example, a polymerization initiator or rate modifier is loaded on the flexible material, so that the initiator or rate modifier provides the desired initiation or rate modification effect to a subsequently applied polymerizable adhesive composition. The polymerization initiator or rate modifier can be immobilized on the flexible material, so that the initiator or rate modifier does not become detached from the flexible material and its residues dispersed in the resultant polymeric material. Alternatively, for example, the polymerization initiator or rate modifier may be initially attached to the flexible material, but only in such a manner that it becomes mobilized or solubilized by a subsequently applied polymerizable adhesive composition and dispersed in the resultant polymeric material.

Suitable initiators are described, for example, in U.S. Pat. Nos. 5,928,611 and 6,620,846, and U.S. Patent Application No. 2002/0037310, all are incorporated herein by reference in its entirety. Quaternary ammonium chloride and bromide salts useful as polymerization initiators are particularly suitable. By way of example, quaternary ammonium salts such as domiphen bromide, butyrylcholine chloride, benzalkonium bromide, acetyl choline chloride, among others, may be used. Also, benzalkonium or benzyltrialkyl ammonium halides such as benzyltrialkyl ammonium chloride may be used. When used, the benzalkonium halide may be in its unpurified state, which comprises a mixture of varying chain-length compounds, or it can be any suitable purified compound including those having a chain length of from about 12 to about 18 carbon atoms, including but not limited to C12, C13, C14, C15, C16, C17, and C18 compounds. By way of example, the initiator may be a quaternary ammonium chloride salt such as benzyltrialkyl ammonium chloride (BTAC).

If desired, a combination of chemical substances can also be provided on the flexible material, to provide multiple effects. For example, as described above, a first chemical species (such as a polymerization initiator or rate modifier) can be immobilized on the flexible material, while a second, different chemical species (such as a bioactive material) can be detachably attached to the flexible material. Other combinations of chemical species and resultant effects are also envisioned by the present invention.

When present in or on the flexible material, the chemical substances (i.e., polymerization initiator, rate modifier, and/or bioactive materials, or other additives), can be incorporated in or on the flexible material in any suitable manner. For example, the chemical substance can be added to the flexible material by contacting the flexible material with a solution, mixture, or the like including the chemical substances. The chemical substance can be added to the flexible material, for example, by dipping, spraying, roll coating, gravure coating, brushing, vapor deposition, or the like. Alternatively, the chemical substance can be incorporated into or onto the flexible material during manufacture of the flexible material, such as during molding or the like of the flexible material.

The chemical substance can be present in or on the flexible material in any suitable concentration and manner. For example, the chemical substance can be applied in a uniform manner to the flexible material, such that there is a substantially uniform concentration of the chemical substance across the flexible material. Alternatively, the chemical substance can be applied such that a concentration gradient exists across or through the flexible material. For example, a greater or smaller concentration of the chemical substance could exist at the center or edges of the flexible material, or a greater or smaller concentration of the chemical substance could be applied on one side of the flexible material as compared to an opposite side. Further, the chemical substance can be applied in a uniform manner to the flexible substrate, or it can be applied in a non-uniform random or patterned manner (such as lines, dots, concentric circles, or the like).

Other chemical substances that can be present in or on the flexible material include, but are not limited to, any suitable and preferably compatible additive that enhances performance of the composite structure. Such additional chemical substances can be bioactive or non-bioactive. Suitable other chemical substances thus include, but are not limited to, colorants (such as inks, dyes and pigments), scents, protective coatings that do not chemically detach, temperature sensitive agents, drugs, and the like.

The polymerization initiator or rate modifier being loaded on the flexible material provides a number of advantages. For example, the structure of the present invention, in embodiments, allows for tailoring of the setting or polymerization time of the applied polymerizable adhesive composition. For example, as is well known in the art, the type and/or concentration of initiator that is applied to the flexible material can be selected so as to provide faster or slower polymerization time. For example, the concentration of polymerization initiator or rate modifier can be increased to provide a faster polymerization time, or can be decreased to provide a slower polymerization time.

Other properties of the polymerization can also be adjusted, in embodiments. For example, the polymerization can be made more uniform than previously possible, at least because the polymerization initiator or rate modifier is generally more uniformly applied to the flexible material. The cure temperature of the polymerizable adhesive composition can also be more easily tailored. For example, the initiator type and/or concentration can be selected to provide a desired polymerization or set time, while not generating excessive heat that could damage the underlying application surface.

Typically, when cyanoacrylate monomers are employed, the adhesive seal formed will remain on the skin for about 7 to about 14 days. However, the monomers may be selected to provide a longer or shorter time on the skin, as desired for a particular use. The fixation devices and systems described may be sterilized by means as known to those of skill in the art.

Various other features and advantages of the embodiments of the invention will be apparent from the following description of exemplary embodiments and figures.

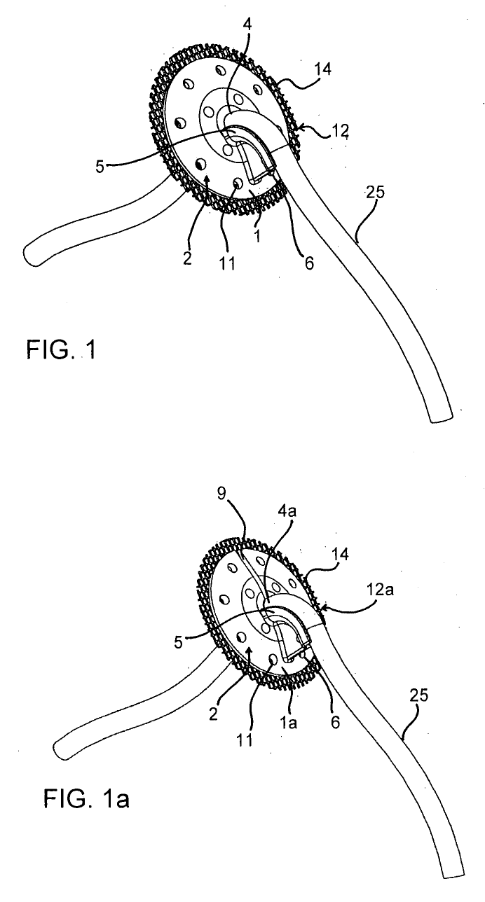

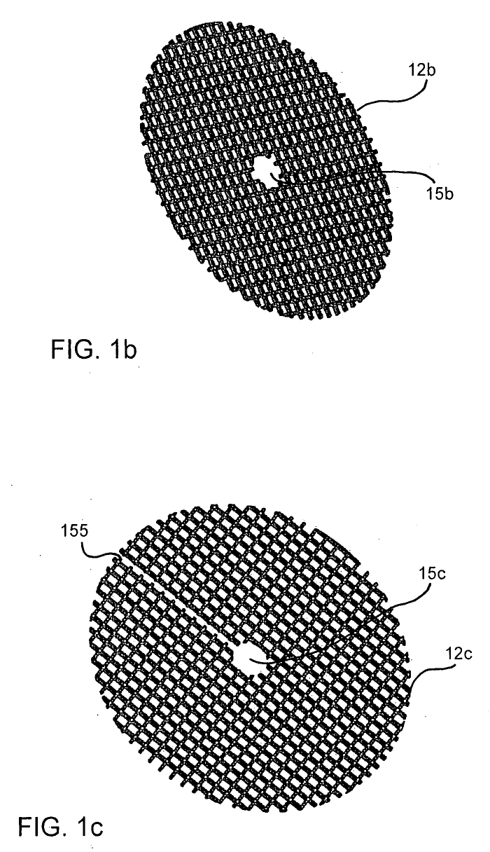

FIG. 1 is a perspective view of an embodiment of a device for the fixation of a medical article including a base member 1 and a flexible member 12. The base member 1 has an upper base member surface 2 and a lower base member surface (not shown). The flexible member 12 has an upper flexible member surface 14 and a lower flexible member surface (not shown). The lower base member surface is affixed to the upper flexible member surface 14. The base member has an opening 4 through the base member from the lower base member surface to the upper base member surface through which the medical article 25 passes. An arcuate channel 5 is proximally integral with the upper base member surface extending distally from the opening in the upper base member surface. The arcuate channel includes a groove 6 in which the medical article sits upon use. The base member further includes one or more polymerizable adhesive composition application holes 11 extending from the upper base member surface through the lower base member surface. A polymerizable adhesive composition may be permeated through at least a portion of the flexible member by application of the polymerizable adhesive composition through the polymerizable adhesive composition application holes in the base member.

FIG. 1a is a perspective view of an embodiment of a device for the fixation of a medical article including a base member 1a and a flexible member 12a. The fixation device includes a passageway 9 in the base member 1a and the flexible member 12a from opening 4a to the perimeter of base member 1a.

FIG. 1b is a perspective view of an embodiment of flexible member 12b prior to affixing the flexible member to the base member 1. Flexible member 12b includes aperture 15b. FIG. 1c is a perspective view of an embodiment of flexible member 12c having a slit 155 from a central aperture 15c to the outer perimeter, which slit may correspond to a passageway in the base member, where present, for example, passageway 9 in FIG. 1a.

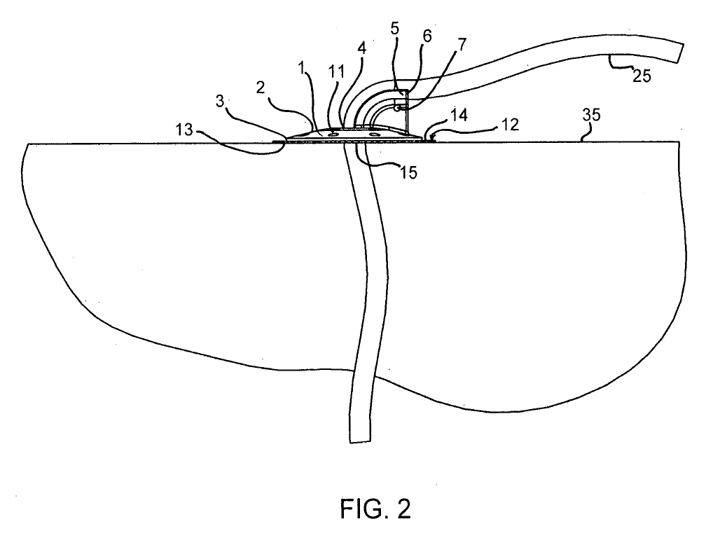

As shown in FIG. 2, the device in the embodiment of FIG. 1 may be used to affix an elongated medical article to the skin. As shown, a medical article 25 is placed in the body extending outwardly through the skin 35 and through the fixation device of FIG. 1. The medical article extends through the flexible member 12 to which is affixed base member 1. The flexible member 12 has a lower flexible member surface 13 and an upper flexible member surface 14. The base member 1 has an upper base member surface 2 and a lower base member surface 3. The flexible member includes an aperture 15 which corresponds to an opening 4 in the base member. The medical article extends outwardly from the body through the skin 35 and through the corresponding opening 4 and aperture 15 in the base member and the flexible member, respectively. The base member includes an arcuate channel 5 proximally integral with the upper base member surface extending distally from the opening in the upper base member surface. The arcuate channel 5 includes a groove 6 through which the medical article extends. The arcuate channel also includes a slot 7 for placing an attachment member for affixing the medical article to the arcuate channel during use. The base member further includes one or more polymerizable adhesive composition application holes 11 extending from the upper base member surface through the lower base member surface wherein the polymerizable adhesive composition is permeated through at least a portion of the flexible material by application of the polymerizable adhesive composition through the polymerizable adhesive composition application holes in the base member.

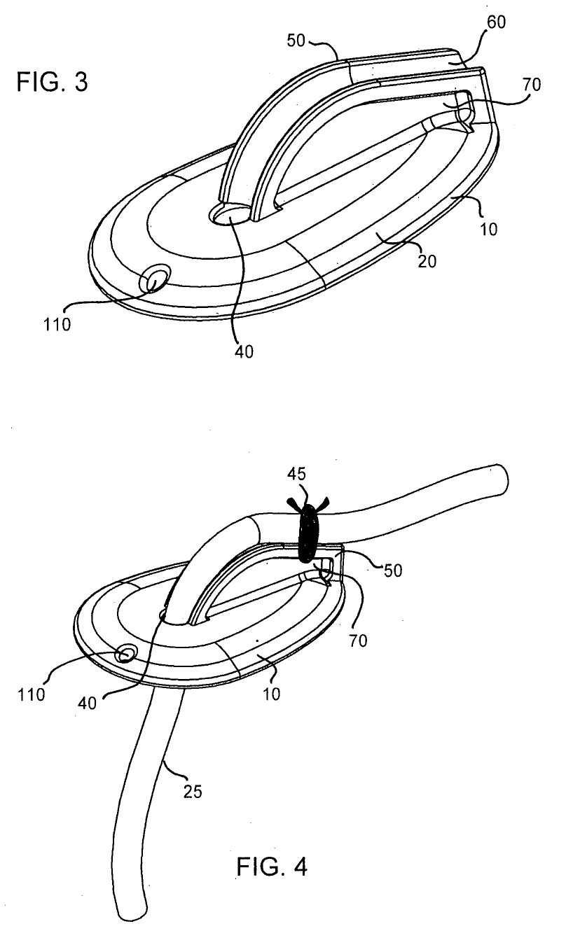

A second embodiment of a fixation device is shown in FIGS. 3-6. Base member 10 is shown in FIG. 3 from a perspective top view. Base member 10 includes an upper base member surface 20, an opening 40 through the base member from the lower base member surface (not shown) to the upper base member surface 20, and a polymerizable adhesive composition application hole 10 extending from the upper base member surface 20 through the lower base member surface. The base member 10 further has an arcuate channel 50 proximally integral with the upper base member surface 20 extending distally from the opening 40 in the upper base member surface 20. The arcuate channel includes a groove 60 for the medical article to be affixed to a patient's body using the fixation device. A slot 70 for an attachment member is included in the arcuate channel. FIG. 4 is an illustration of the embodiment of FIG. 3 including the medical article 25 and an attachment member (a suture) 45.

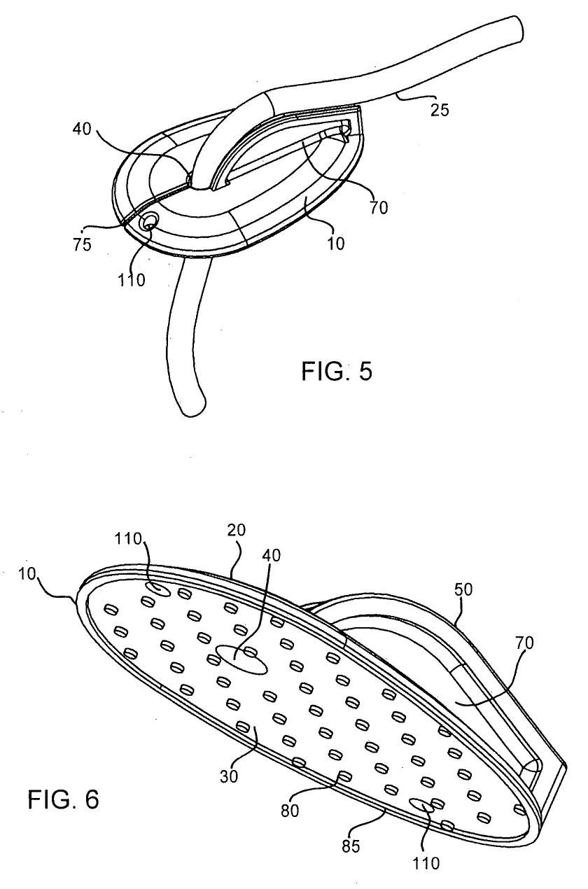

FIG. 5 illustrates an embodiment wherein a passageway 75 is present in the base member from the opening 40 through the base member to the perimeter of the base member. The passageway 75 permits the medical article 25 to be placed in the opening 40 of the base member 10 by sliding the medical article through the passageway rather than threading the medical article 25 through the opening 40. This embodiment permits the fixation device to be placed over the medical article after the medical article is inserted into the patient without threading the fixation device over the entire end of the medical article. This embodiment also permits the fixation device to be removed by sliding the medical article 25 through the passageway 75. The passageway 75 may be sized for the particular use based on the size and flexibility of the medical article and the flexibility of the material used for the base member. Base member 10 will include one or more polymerizable adhesive composition application holes 110 extending from the upper base member surface through the lower base member surface. The polymerizable adhesive composition application hole 110 may be placed in the base member in any location desired for a particular use or purpose.

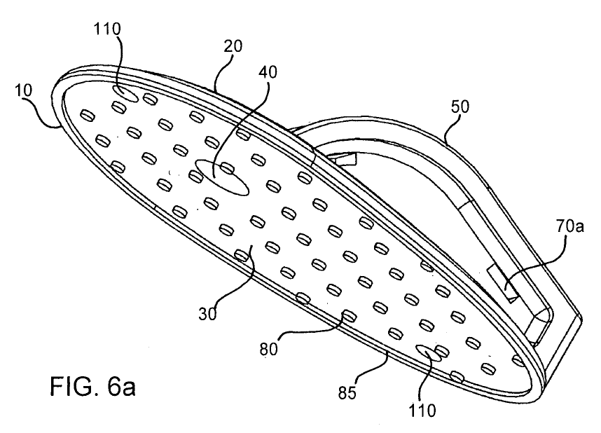

FIG. 6 illustrates an embodiment of the fixation device as seen from underneath the device. As shown, the base member 10 has an upper base member surface 20 and a lower base member surface 30. An opening 40 extends through the base member from the lower base member surface 30 to the upper base member surface 20. An arcuate channel 50 proximally integral with the upper base member surface extends distally from the opening 40 in the upper base member surface 20. The arcuate channel 50 includes a slot 70 for placement of an attachment member. The lower base member surface 30 includes a wall portion 85 extending outwardly from and surrounding the perimeter of the lower base member surface. Protrusions 80 project from the lower base member surface 30. Polymerizable adhesive composition application holes 110 extend from the upper base member surface 20 through the lower base member surface 30. When placed on the skin of a patient, the wall portion 85 and protrusions 80 contact the skin forming a gap between the lower base member surface and the skin of the patient. A polymerizable adhesive composition may be applied through the polymerizable adhesive composition application holes 110. The adhesive composition spreads under the base member, polymerizing and bonding the fixation device to the skin. The wall portion 85 prevents the adhesive composition from flowing out from underneath the fixation device.

FIG. 6a illustrates the area under the arcuate channel provided with slot 70a for placement of an attachment member.

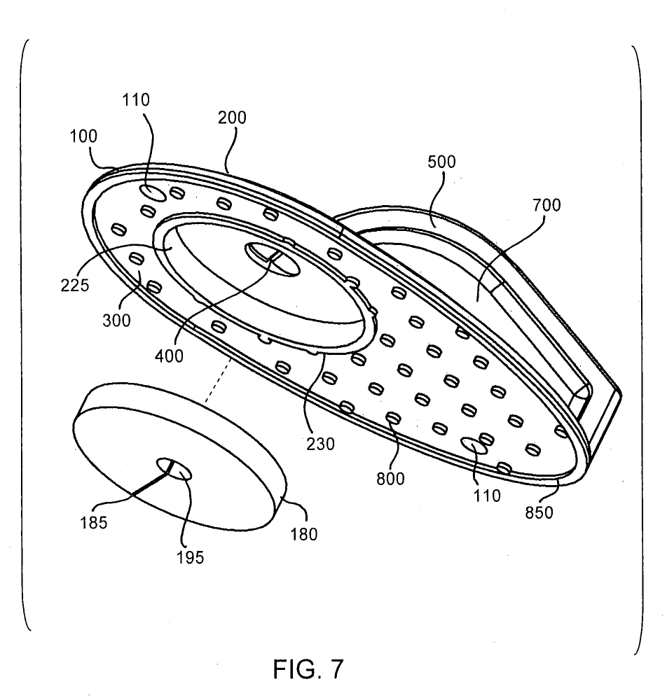

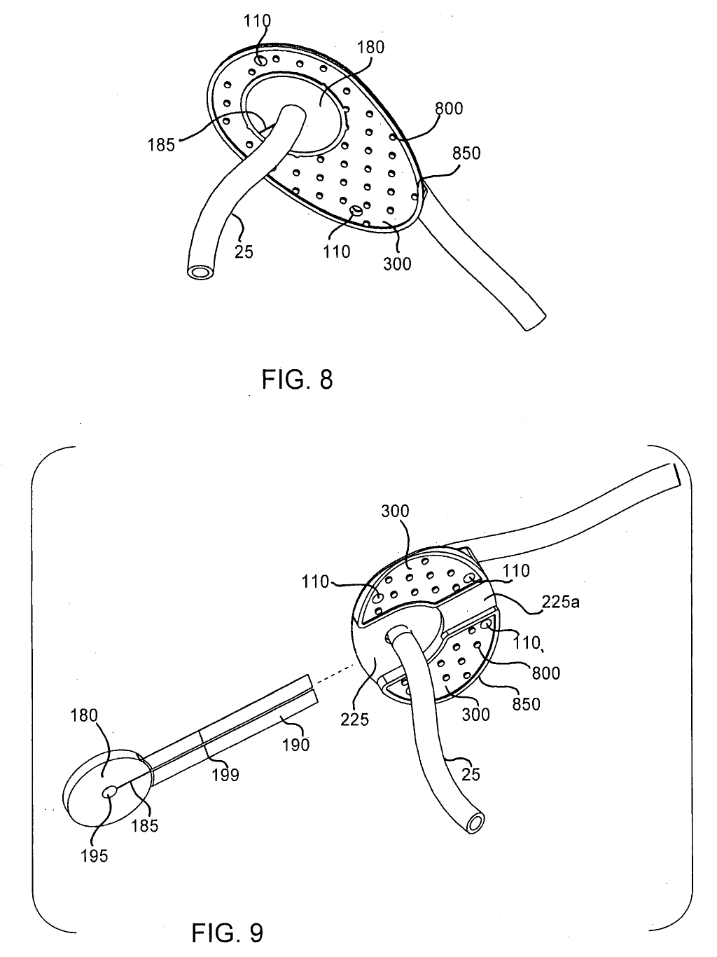

FIG. 7 and FIG. 8 illustrate another embodiment of the fixation device. As shown, the fixation device includes base member 100 with upper base member surface 200 and lower base member surface 300. The base member 100 includes opening 400 through the base member from the lower base member surface 300 to the upper base member surface 200. The base member has protrusions 800 projecting from the lower base member surface 300 and a wall portion 850 extending outwardly from and surrounding the perimeter of the lower base member surface 300. An arcuate channel 500 is proximally integral with the upper base member surface 200 extending distally from the opening 400 in the upper base member surface 200. The arcuate channel 500 includes a slot 700 for placement of an attachment member. Polymerizable adhesive composition application holes 110 extend from the upper base member surface 200 through the lower base member surface 300. The base member further includes a recessed cavity 225 which surrounds opening 400. The recessed cavity is sized for placement of absorptive foam member 180. A lip 230 surrounds the recessed cavity and extends outwardly from the edge of the recessed cavity. The lip extends outwardly from the edge of the recessed cavity the same distance as the wall portion 850 and the protrusions 800. In use, the lip, the wall portion and protrusions rest on the skin forming a gap between the lower base member surface 300 and the skin of the patient.

The recessed cavity 225 is sized for containment or placement of an absorptive foam member. Absorptive foam member 180 has a slit 185 for placement over a medical article, if needed, and a gap 195 which corresponds to the opening 400 through the base member 100 from the lower base member surface 300 to the upper base member surface 200. As shown in FIG. 8, in use, medical article 25 is placed through opening 400 and gap 195 and the absorptive foam member 180 is positioned within the recessed cavity.

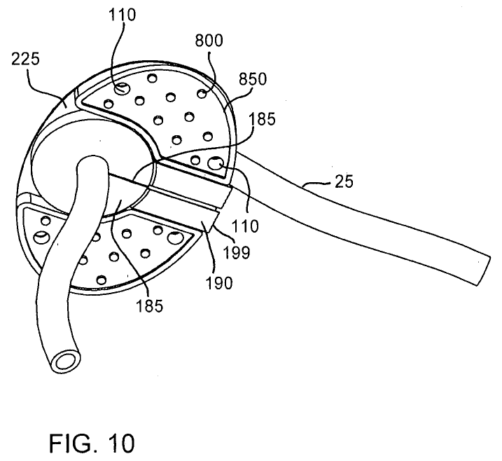

FIG. 9 and FIG. 10 illustrate an embodiment wherein the absorptive foam member includes pull tabs 190 for placement in the fixation device. In this embodiment, the absorptive foam member may be installed under the fixation device after placement of the fixation device on the skin or tissue of a patient. The pull tab 190 may have perforations 199 for removal of the pull tabs once the absorptive foam member is placed in the recessed cavity 225. The pull tab 190 may be adhered to the top of the absorptive foam member using pressure sensitive adhesive or by other means known to those of skill in the art. In use, the fixation device is installed over the medical article and adhered to a patient with a polymerizable adhesive composition. The absorptive foam member is installed by sliding pull tab 190 under the base member through the recessed cavity 225 and extended recessed cavity 225a. As shown, the base member includes recessed cavity 225 and extended recessed cavity 225a from recessed cavity 225 to the perimeter of base member. The pull tabs 190 are fed out the back of the fixation device and pulled until the absorptive foam member is positioned in the recessed cavity. The pull tab 190 may be removed by tearing at the perforations 199.

Extended recessed cavity 225a as shown is a shallower recess than the recessed cavity 225 for passing pull tab 190 underneath the base member 100. As shown in FIG. 9, the lower base member surface 300 is divided into two segments by the recessed cavity 225 and extended recessed cavity 225a. In this embodiment, the wall portion 850 surrounds the lower base member surface and the recessed cavity 225 and associated extended recessed cavity 225a, thus serving as the lip for the recessed cavity (shown as 230 in FIG. 7). FIG. 10 illustrates the embodiment of FIG. 9 wherein the absorptive foam member has been positioned in the base member and the pull tab has been removed at the perforation 199.

While the invention has been described with reference to preferred embodiments, the invention is not limited to the specific examples given, and other embodiments and modifications can be made by those skilled in the art without departing from the spirit and scope of the invention.

1. A device for securing an elongated medical article to the skin of a patient comprising:
a) a flexible member comprising a flexible material and a polymerization initiator or rate modifier permeated throughout at least a portion of said flexible material, said flexible member having a lower flexible member surface, an upper flexible member surface and an aperture through the flexible member; and
b) a base member comprising an upper base member surface,
a lower base member surface affixed to the upper flexible member surface,
an opening through the base member from the lower base member surface to the upper base member surface corresponding to the aperture in the flexible member,
fluid communication between the upper base member surface and the lower base member surface, and
an arcuate channel projecting from the upper base member surface in proximity to the opening through the base member.

2. The device of claim 1 wherein the fluid communication between the upper base member surface and the lower base member surface comprises one or more polymerizable adhesive composition application holes.

3. The device of claim 1 wherein the flexible material is a mesh.

4. The device of claim 1 further comprising an attachment member for attaching an elongated medical article to the arcuate channel on the base member.

5. The device of claim 1 wherein the flexible member further comprises a slit from the aperture to the perimeter of the flexible member and the base member further comprises a passageway from the opening to the perimeter of the base member, wherein the slit and the passageway are in corresponding position.

6. A fixation system kit for securing an elongated medical article to the skin of a patient comprising:
a) a flexible member comprising a lower flexible member surface, an upper flexible member surface and an aperture through the flexible member;
b) a base member comprising an upper base member surface,
a lower base member surface affixed to the upper flexible member surface,
an opening through the base member from the lower base member surface to the upper base member surface corresponding to the aperture in the flexible member, and
an arcuate channel projecting from the upper base member surface in proximity to the opening through the base member; and
c) an effective amount of polymerizable adhesive composition.

7. The fixation system kit of claim 6 wherein the base member further comprises fluid communication between the upper base member surface and the lower base member surface comprising one or more polymerizable adhesive composition application holes.

8. The fixation system kit of claim 6 wherein the flexible material is a mesh.

9. A device for securing an elongated medical article to the skin of a patient comprising:
a base member comprising an upper base member surface,
a lower base member surface,
an opening through the base member from the lower base member surface to the upper base member surface,
fluid communication between the upper base member surface and the lower base member surface,
an arcuate channel projecting from the upper base member surface in proximity to the opening through the base member, and
a plurality of protrusions projecting from the lower base member surface.

10. The device of claim 9 further comprising a wall portion extending outwardly from and at least partially surrounding the perimeter of the lower base member surface.

11. The device of claim 9 further comprising an attachment member for attaching an elongated medical article to the arcuate channel.

12. The device of claim 11 wherein the arcuate channel comprises a slot for the attachment member.

13. The device of claim 9 wherein the base member further comprises a passageway radially extending through the base member to the perimeter of the base member.

14. The device of claim 9 wherein the base member further comprises a recessed cavity in the lower base member surface around the opening through the base member from the lower base member surface to the upper base member surface.

15. The device of claim 14 wherein an absorptive foam member is secured in the recessed cavity.

16. The device of claim 15 wherein the absorptive foam member comprises a hydrophilic polyurethane absorptive foam and an antimicrobial agent.

17. The device of claim 14 wherein the recessed cavity is extended to the perimeter of the base member.

18. A fixation system kit for securing an elongated medical article to the skin of a patient comprising:
a) a base member comprising an upper base member surface,
a lower base member surface having a plurality of protrusions integral with and projecting from the lower base member surface which contact the skin of the patient forming a gap between the lower base member surface and the skin of the patient,
an opening through the base member from the lower base member surface to the upper base member surface for receiving the elongated medical article,
fluid communication between the upper base member surface and the lower base member surface for application of a polymerizable adhesive composition to the gap between the lower base member surface and the skin of the patient, and
an arcuate channel projecting from the upper base member surface in proximity to the opening through the base member;
b) an amount of polymerizable adhesive composition effective to affix the base member to the skin; and
c) an attachment member for attaching the elongated medical article to the arcuate channel on the base member.

19. The fixation system kit of claim 18 wherein the polymerizable adhesive composition comprises a cyanoacrylate monomer.

20. The fixation system kit of claim 18 wherein the base member further comprises a recessed cavity in the lower base member surface around the opening through the base member from the lower base member surface to the upper base member surface and the fixation system further comprises an absorptive foam member which may be secured in the recessed cavity.
